(19) United States
(12) Patent Application Publication
(10) Pub. No.: US 2009/0264308 A1
(43) Pub. Date: Oct. 22, 2009

(54) BIOSENSOR SOLID SUBSTRATE WITH INTEGRATED TEMPERATURE CONTROL AND A METHOD TO MAKE THE SAME

(75) Inventors: Dirk Jan Broer, Eindhoven (NL); Roel Penterman, Eindhoven (NL); Ralph Kurt, Eindhoven (NL); Emiel Peeters, Eindhoven (NL); Hendrik Roelof Stapert, Eindhoven (NL); Anke Pierik, Eindhoven (NL); Aleksey Kolesnychenko, Eindhoven (NL); Hendrik De Koning, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/301,645

(22) PCT Filed: May 22, 2007
(86) PCT No.: PCT/IB07/51935

§ 371 Date: Nov. 20, 2008

(30) Foreign Application Priority Data
May 24, 2006 (EP) 06114503.3

(51) Int. Cl. C40B 30/04; C12Q 1/68; C12M 1/34; C40B 40/06

(52) U.S. Cl. 506/9; 435/6; 435/287.2; 506/16

(57) ABSTRACT

This invention provides a biosensor solid or hydrogel substrate comprising one or more temperature indicating agents, each of said one or more temperature indicating agents operating by changing its optical properties and being deposited in and/or at the surface of the biosensor solid or hydrogel substrate as one or more layers and/or spots

### FIELD OF THE INVENTION

The present invention relates to a sensor substrate such as a biosensor solid or hydrogel substrate for the analysis of a sample fluid suspected of containing one or more target analyte molecules such as biological compounds. In particular, the present invention permits an inexpensive, fast and precise measure of the temperature and the distribution thereof at the level of the biosensor solid substrate. The present invention also relates to a biosensor device comprising a biosensor solid substrate with integrated temperature monitoring and/or control means. Such biosensor substrates, and devices incorporating them, are useful as analytical and diagnostic tools in the fields of human and veterinary medicine, among others. In particular, the present invention relates to a method of analysis of a sample fluid suspected of containing one or more analyte molecules such as target biological compounds, which method can be used for molecular diagnostic tests, e.g. for measuring the presence of infectious disease pathogens and resistance genes.

### BACKGROUND TO THE INVENTION

The presence and concentration of specific target biological compounds, such as but not limited to, DNA, RNA or proteins, in a sample fluid containing one or more other molecules can be determined by using the complex binding of these target biological compounds with probes. In the case of the traditional Western/Southern/Northern Blot, the target biological compound is immobilized on the blot surface and subsequently detected by a soluble probe. For ELISA (enzyme-linked immunosorbent assay) or microarray based tests, the probes are immobilized instead. In the microarray technique, a set of specific probes, each of which being chosen in order to interact specifically (i.e. hybridize) with one particular target biological compound, are immobilized at specific locations of a biosensor solid substrate. On the other hand, the target biological compounds are labeled by a detectable label molecule (e.g., but not limited to, a fluorophore or a magnetic bead). By contacting said solid substrate with the sample fluid, the target biological compounds are fixed at the locations corresponding to their specific probes. The detection of the target biological compounds in the sample fluid is then operated via the localization of the signals produced by the detectable molecules bound to the target biological compounds.

Hybridization being a temperature-dependant phenomenon, temperature control provides significant advantages in this technology, e.g. for nucleic acid analyses. WO 03/004162 discloses a biosensor device for performing hybridization assays at various temperatures (e.g. 20 to 46±2° C.). The device includes a system for controlling the temperature of a test fluid (e.g. a sample fluid) by heat transfer between this test fluid and a thermal fluid (e.g. water or ethylene glycol) delivered from a conventional thermostatic fluid bath and pump system. This thermal fluid is circulating in a separate circuit in the vicinity of the test fluid. This method of control relies on temperature measurements at the level of the thermostatic fluid bath while the part of the device where temperature control is most critical is the biosensor solid substrate. This prior art therefore fails to permit precise control of the temperature, and distribution thereof, over the biosensor solid substrate surface. There is a need in the art for improving, in a cost-effective manner, the temperature control, especially up to the level where substantially a homogeneous temperature within a few tens of degrees Celsius can be achieved. There is a need in the art for a precise and reliable method and device to measure the temperature, and its distribution, directly at the level of the biosensor solid substrate. There is also a need in the art for a method of making such improved biosensor devices, wherein said method is easy to perform and does not significantly increase the cost of said device.

An object of the present invention is to provide a good sensor substrate such as a biosensor solid or hydrogel substrate, and a method of producing the same, which can easily be incorporated into a sensor such as a biosensor device and which permits to monitor and/or control the temperature of the substrate during processing. An advantage of the present invention is that the temperature can be monitored and/or controlled at which a preparation step, an amplification step or a detection step is performed on a biosensor substrate. The present invention also relates to a device incorporating this sensor substrate, e.g. biosensor solid or hydrogel substrate, as well as to a method of analysis of a sample fluid suspected of containing one or more analyte molecules such as target biological compounds.

### SUMMARY OF THE INVENTION

Broadly speaking, the invention is based on the finding that the temperature of a sensor substrate such as a biosensor solid or hydrogel substrate can be monitored and/or controlled in a precise, fast and inexpensive way by depositing one or more temperature indicating agents into and/or at the surface of the sensor substrate such as a biosensor solid or hydrogel substrate as one or more layers and/or spots. The deposited agents may operate by changing their optical properties, dependent upon the temperature.

This construction of the sensor substrate such as the biosensor solid or hydrogel substrate has the advantage to permit monitoring of the temperature of the substrate itself. A biosensor device incorporating such a biosensor solid or hydrogel substrate is useful for performing an easy, accurate and inexpensive analysis of a sample fluid suspected of containing one or more target biological compounds.

The solid or hydrogel substrate can be porous, i.e. is made of solid material (e.g. nylon fibers) and is porous.

### DETAILED DESCRIPTION OF THE DRAWINGS

The present invention, its embodiments and advantages will be described with reference to the following drawings.

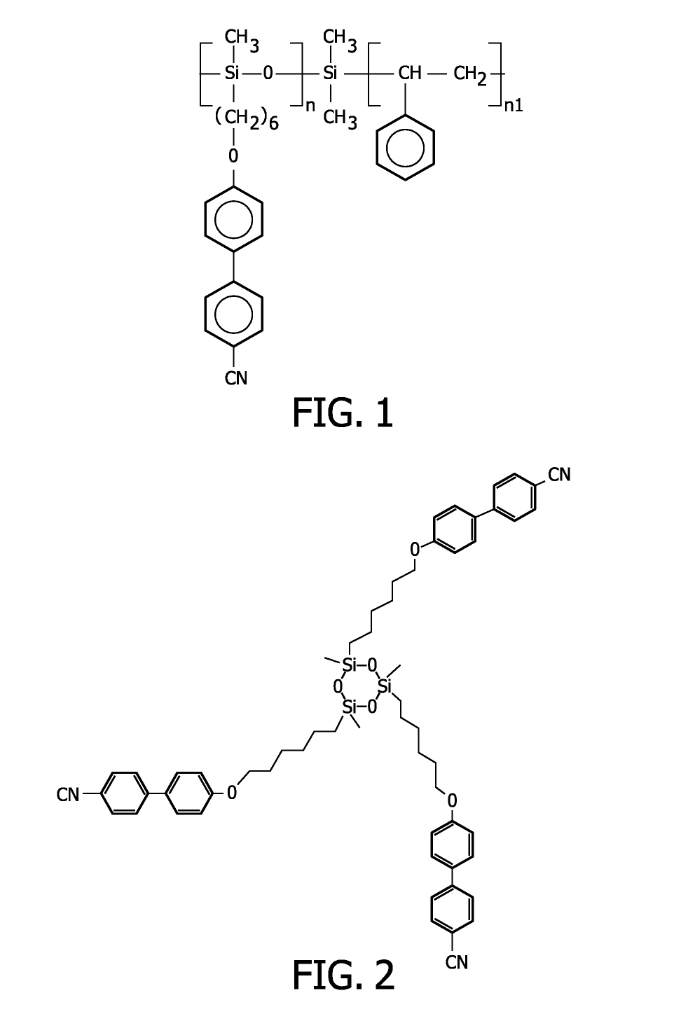

FIG. 1 is the chemical structure of a particular example of a polymeric liquid crystal usable as a temperature indicating agent according to an embodiment of the present invention.

FIG. 2 is the chemical structure of a particular example of a siloxane ring liquid crystal usable as a temperature indicating agent according to an embodiment of the present invention.

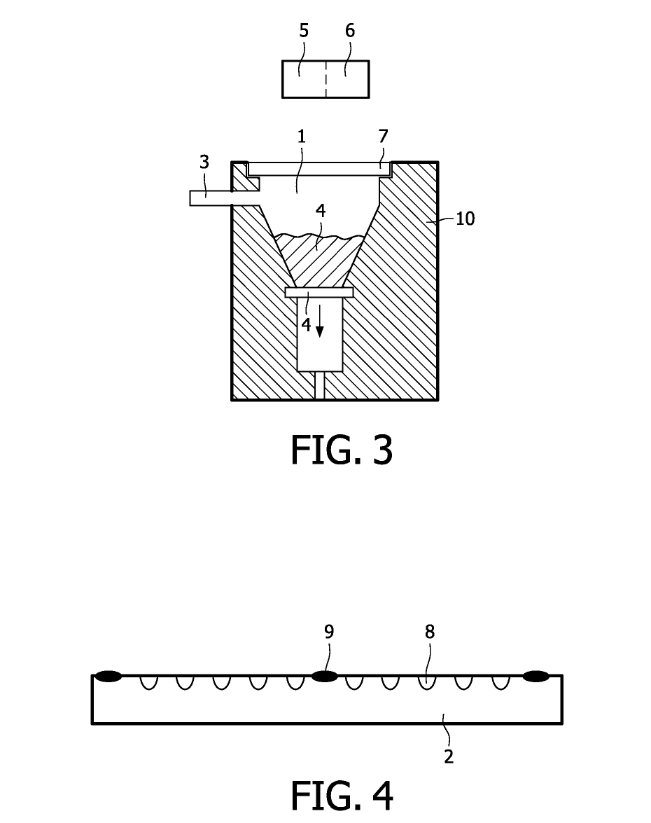

FIG. 3 is a schematic view of a cross-section of a biosensor device according to an embodiment of the present invention.

FIG. 4 is a schematic view of a cross-section of a biosensor solid or hydrogel substrate according to an embodiment of the present invention.

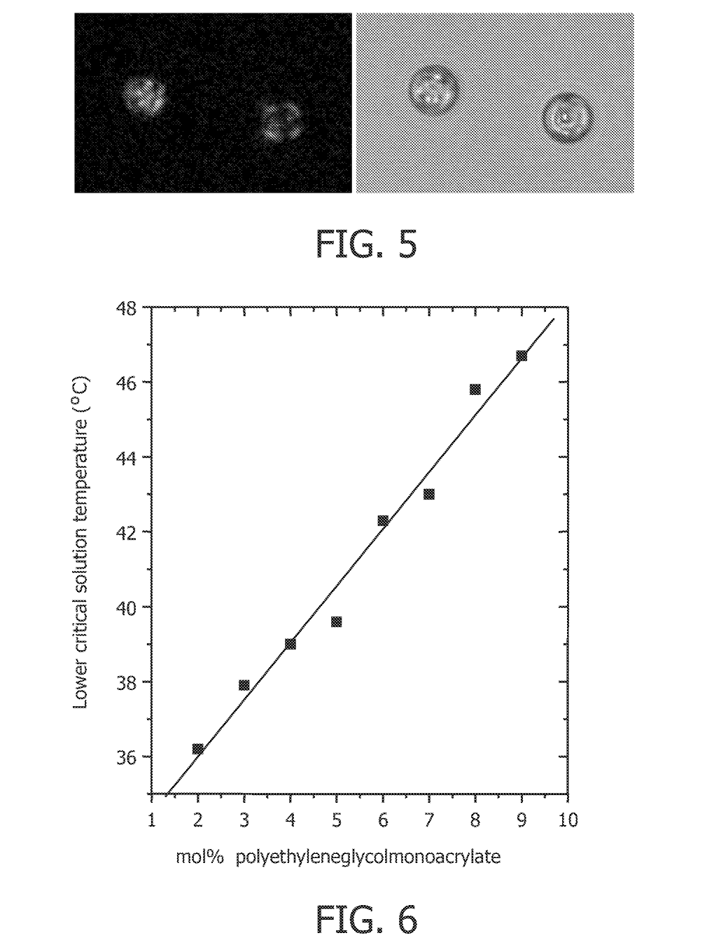

FIG. 5 represents two photographs showing LC-filled polymer capsules usable in embodiments of the present invention.

FIG. 6 is a graph of the observed lower critical solution temperature (LCST) of co-polymers NIPA-PEGA versus the ratio PEGA/NIPA

### DETAILED DESCRIPTION

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

As used herein, and unless stated otherwise, the term <<biosensor>>, when applied to a substrate, designates a substrate which purpose is to enable the detection of the presence, absence or concentration of one or more target biological compounds by any suitable method. Exemplary but non-limiting methods are:

Retention or immobilizing at specific locations into or onto said substrate either the one or more target biological compounds themselves (as would be the case for a biosensor substrate used in Western/Southern/Northern Blot) or one or more probes, each being capable to bind specifically with one of the one or more target biological compounds (as would be the case in an ELISA or in a microarray assay) and,

binding specifically at least one of the immobilized member of the couples probes/target biological compounds with the complementary member of this couple still present in solution.

amplification such as PCR, rtPCR (real time), RT-PCR (reverse transcription) or QPCR (quantitative) on a chip,

electrophoresis in a microfluidic system combined with e.g. laser induced fluorescence (LIF).

As used herein, and unless stated otherwise, the term <<microarray assay >> designates an assay wherein a sample fluid, preferably a biological fluid sample (optionally containing minor amounts of solid or colloid particles suspended therein), suspected to contain target biological compounds is contacting (i.e. flowing over or flowing through) a biosensor solid substrate containing a multiplicity of discrete and isolated regions across a surface thereof, each of said regions having one or more probes applied thereto and each of said probes being chosen for its ability to bind specifically with a target biological compound.

As used herein, and unless stated otherwise, the term <<target biological compound>> designates a biological molecular compound fixed as a goal or point of analysis. It includes biological molecular compounds such as, but not limited to, nucleic acids and related compounds (e.g. DNAs, RNAs, oligonucleotides or analogs thereof, Polymerase chain reaction (PCR) products, genomic DNA, bacterial artificial chromosomes, plasmids and the like), proteins and related compounds (e.g. polypeptides, peptides, monoclonal or polyclonal antibodies, soluble or bound receptors, transcription factors, and the like), antigens, ligands, haptens, carbohydrates and related compounds (e.g. polysaccharides, oligosaccharides and the like), cellular fragments such as membrane fragments, cellular organelles, intact cells, bacteria, viruses, protozoa, and the like.

As used herein, and unless stated otherwise, the term <<probe >> designates a biological agent being capable to bind specifically with a <<target biological compound>> when put in the presence of or reacted with said target biological compound, and used in order to detect the presence of said target biological compound. Probes include biological molecular compounds such as, but not limited to, nucleic acids and related compounds (e.g. DNAs, RNAs, oligonucleotides or analogs thereof, PCR products, genomic DNA, bacterial artificial chromosomes, plasmids and the like), proteins and related compounds (e.g. polypeptides, monoclonal antibodies, receptors, transcription factors, and the like), antigens, ligands, haptens, carbohydrates and related compounds (e.g. polysaccharides, oligosaccharides and the like), cellular organelles, intact cells, and the like. Probes may also include specific materials such as certain biopolymers to which target compounds bind.

As used herein, and unless stated otherwise, the term <<label >> designates a biological or chemical agent having at least one physical property (such as, but not limited to, radioactivity, optical property, magnetic property) detectable by suitable means so as to enable the determination of its spatial position and/or the intensity of the detectable physical property such as, but not limited to, luminescent molecules (e.g. fluorescent agents, phosphorescent agents, chemiluminescent agents, electroluminescent agents, bioluminescent agents and the like), colored molecules, molecules producing colors upon reaction, enzymes, magnetic beads, radioisotopes, specifically bindable ligands, microbubbles detectable by sonic resonance and the like.

As used herein, and unless stated otherwise, the term <<tag >> designates the action of bringing a label in the presence of a probe, or linking or interacting (e.g. reacting) a label with a probe.

As used herein, and unless stated otherwise, the term “hydrogel” designates a hydrophilic polymer network capable of swelling in water and other aqueous media or polar solvents (e.g. alkanols such as ethanol, methanol or isopropanol), and able of retaining large volumes of water and/or solvent in the swollen state (50% or more water, preferably 70% or more water, most preferably 90% or more water). In the swollen state, hydrogels consist of a three-dimensional network of polymer chains that are solvated by water and/or polar solvent molecules while the chains are chemically or physically linked to each other, thus preventing the polymer network from dissolving in the aqueous or polar organic environment.

As used herein and unless stated otherwise, the terms “crosslink density” δ_{X} is defined as follow:

$${δ}_{X} = \frac{X}{L + X}$$

wherein X is the mole fraction of polyfunctional monomers and L is the mole fraction of linear chain (=non polyfunctional) forming monomers. In a linear polymer δ_{X}=0, in a fully crosslinked system δ_{X}=1.

This invention is based on depositing into and/or at the surface of a sensor substrate such as a biosensor solid or hydrogel substrate, as one or more layers and/or spots, at least one agent which indicates temperature through a change in a physical property, e.g. an optical property, electrical property, magnetic property, a mechanical force or pressure or a change in shape. Examples of changes of optical properties that can indicate a temperature change within the meaning of the present invention include but are not limited to changes in color, absorption, transmission, spectrum, polarization, index of refraction, scattering, measured light intensity, excitation, fluorescence and the like.

As an optional feature, the spots may have their smaller dimension ranging from 1 μm to 1 mm, preferably 5-500 μm, most preferably 10-300 μm.

As another optional feature, the spots may be circular, spherical, rod-like or pillar like among others.

Preferably, this change in the physical property, e.g. the optical property, is detectable when the temperature of the substrate, e.g. solid or hydrogel substrate having temperature indicating agents, is within a temperature range comprised between about 20° C. and about 95° C. Also preferably, this change in a physical property, e.g. optical property preferably occurs within not more than about 3° C., more preferably within not more than 1° C. This feature is advantageous because it permits to accurately assess the temperature of the sensor substrate such as the biosensor solid or hydrogel substrate.

As an optional feature, the temperature indicating agent may gradually change its optical property over a wide range of temperature. For instance, thermo-responsive fluorescent dyes fixed to or in the substrate can be used. Such temperature indicating agents may be advantageous to assess a temperature over a wide temperature range.

As an optional feature, the sensor substrate, e.g. the biosensor solid or hydrogel substrate of this invention may further comprise one or more probes able to each specifically bind one analyte molecule, e.g. a target biological compound. Preferably, said one or more probes form an array of probe locations present in and/or at the surface of the sensor substrate, e.g. the biosensor solid or hydrogel substrate. This feature is advantageous because it allows the simultaneous detection of more than one analyte molecule, e.g. target biological compound.

In embodiments of the present invention, the temperature can be monitored and/or controlled at which the detection of the hybridization of a target biological compound on an immobilized probe (e.g. in the case of ELISA or microarray assay) or the detection of the hybridization of a probe on an immobilized target biological compound (e.g. in the case of Western/Southern/Northern Blot) is performed. In other embodiments of the present invention, a temperature cycle (i.e. a change of temperature according to a pre-defined regime) can be monitored and/or controlled during which an amplification of a target biological compound (e.g. via PCR) is performed. In yet other embodiments of the present invention, the temperature can be monitored and/or controlled at which the preparation of an analyte (e.g. the separation of double stranded DNA) and/or the preparation of a probe (e.g. the activation of a proteinic probe via cleavage) is performed. Other steps in sample preparation which might require accurate temperature control include cell lyses, fluid mixing, filtering (especially affinity reactions and the like), chemical reactions such as binding, coupling or ligand reactions to attach labels, reagents, enzymes, antibodies, primers etc. to specific analytes, and increases or decreases of the concentration of specific components of the analyte sample, among others.

When the temperature indicating agents are deposited as one or more spots, and when one or more probes are present, preferably, the spots are located at places distinct from the location of said one or more probes. This is advantageous because it avoids the contamination of the probes by the temperature indicating agents. It is also advantageous to apply the one or more spots by an ink-jet printing method because it permits the use of a single method to deposit both the temperature indicating agents and the probes with the target biological compounds at the surface of the substrate. However the present invention is not limited to ink jet printing. It is also advantageous to link/fix the temperature indicating agents to the substrate material (e.g. the porous substrate material or hydrogel) as therewith the temperature can be measured very close to the location of interest and migration of the temperature indicating agents is avoided.

Another preferred method to deposit said temperature indicator comprises a photo-patterning step (preferably using UV irradiation through a mask). For instance, in a first step, a film of temperature indicating agent comprising UV graftable moieties, can be applied on the substrate of the biosensor. In a second step, a photografting of the temperature indicating agent may be performed by irradiating UV light through a mask. In a third step, the unreacted temperature indicating agents can be removed. Moreover it is preferred to use photo-polymerization and/or grafting (preferably photo-grafting) of the temperature indicating agents either directly into and/or onto said substrate or indirectly via grafting of a matrix or a capsule comprising the temperature indicating agent.

As another optional feature, at least one of the temperature indicating agents comprise a liquid crystalline material. This feature is advantageous because liquid crystals are available and obtainable within a wide range of liquid to liquid-crystal transition temperatures, especially between 20° C. and 95° C.

As another optional feature, at least one of the one or more temperature indicating agents comprises one or more side-chain liquid crystals with a siloxane polymer backbone and/or one or more liquid crystalline siloxane rings. This optional feature is advantageous because these substances have a low tendency to diffuse into a solid or hydrogel substrate, have a low water uptake tendency and have a relatively sharp liquid-to-liquid crystal transition.

As another optional feature, the sensor substrate, e.g. biosensor solid or hydrogel substrate, according to the present invention may comprise one single substrate material, as an economical alternative, or may be a composite substrate comprising a first substrate material and a second substrate material wherein said second material forms a layer onto the first material. The latter feature has the advantage to benefit from the combination of different properties of two different substrate materials, e.g. the mechanical properties of the first material and the chemical properties of the second material.

As another optional feature, the sensor substrate is a hydrogel substrate having its LCST (lower critical solution temperature) in the range 20 to 95 degree, preferably between 30 and 85 degree.

As another optional feature, at least one of the one or more temperature indicating agents may comprise a coloring agent, preferably fluorescent agents such as e.g. a fluorescent dye or fluorescent beads. This feature is advantageous because it renders the temperature transitions more visible.

As another optional feature, the biosensor substrate may comprise two or more temperature indicating agents, each changing an optical property in a different range of temperature. This is advantageous because it permits to assess the temperature of the substrate over a wider temperature range.

As another optional feature, the one or more temperature indicating agents may comprise both, a temperature responsive polymer, co-polymer or hydrogel and a coloring agent. The coloring agent (e.g. fluorescent beads) may for instance be embedded within the temperature responsive polymer, co-polymer or hydrogel or may be co-polymerized with the temperature responsive polymer, co-polymer or hydrogel or may be deposited on the biosensor substrate before the deposition of the temperature responsive polymer, co-polymer or hydrogel. This is advantageous because this permits the detection of a change of intensity in the optical signal detected (e.g. fluorescence of the fluorescent beads) caused by temperature induced scattering of the temperature responsive polymer, co-polymer or hydrogel.

Another embodiment of the present invention relates to a sensor especially a biosensor device comprising:

a chamber including a substrate such as a biosensor solid or hydrogel substrate comprising one or more temperature indicating agents each operating by changing at least one physical property, e.g. an optical property,

inlet means for introducing a sample fluid suspected to contain one or more analyte molecules such as target biological compounds into the chamber such that the sample fluid contacts the sensor substrate, e.g. biosensor solid or hydrogel substrate,

means for analyzing the sensor substrate, e.g. biosensor solid or hydrogel substrate after the sample fluid has contacted the substrate, e.g. biosensor solid or hydrogel substrate, so as to determine the presence and/or the concentration of the one or more analyte molecules, e.g. target biological compounds on the sensor substrate, e.g. biosensor solid or hydrogel substrate, and

means for analyzing the biosensor solid or hydrogel substrate so as to retrieve temperature-related information from the one or more temperature indicating agents.

As an optional feature, the biosensor device of this invention may further comprise one or more probes able to each specifically bind one of the one or more target biological compounds. As another optional feature, when the temperature indicating agent comprises temperature responsive polymer, co-polymer or hydrogel, the one or more probes may be deposited on the biosensor substrate after the deposition of the temperature responsive polymer, co-polymer or hydrogel. As another optional feature of the biosensor device, the sample fluid may contact the biosensor solid, porous or hydrogel substrate by flowing through it. This feature is advantageous because it reduces the time necessary for the analysis comparatively to flow over techniques.

According to one embodiment of the invention, the means for the determination of the presence of the one or more target biological compounds and the means for retrieving temperature-related information from the one or more temperature indicating agents may be the same means. In this situation, the single means may be an optical means. This is advantageous because it provides both an economical and practical construction of the biosensor device. Said single optical means may includes the cases that the identical illumination/excitation optical means and/or identical optical detection means and/or both identical illumination and optical detection means are use (or at least parts of).

As an optional feature, illumination and/or excitation means may be provided. As another optional feature, when illumination and/or excitation means are provided, they may form part of the same optical device as the means for the determination of the presence of the one or more target biological compounds and the means for retrieving temperature-related information from the one or more temperature indicating agents.

As another optional feature, the biosensor device may further comprise heating means (e.g. a heating means or two or more heating means) for raising the temperature of the sample fluid and/or the biosensor solid or hydrogel substrate. This feature is advantageous because external heating means are no longer required.

As another optional feature, the biosensor device may further comprise cooling means (e.g. a cooling means or two or more cooling means) for lowering the temperature of the sample fluid and/or the biosensor solid or hydrogel substrate. This feature is advantageous because external cooling means are no longer required.

Both heating and/or cooling means may be provided, e.g. a resistive heater or a Peltier element.

Such heating/cooling means (e.g. Peltier elements) can be used to denature and renature DNA in PCR reactions. For the performance of PCR reactions, it is preferable that the device enable the obtaining of accurate temperatures between about 45 and 100° C. It is also preferable that a change in temperature up to 50 degrees can be obtained in about 15 seconds or less.

When the biosensor device further comprises heating means, the biosensor device may further comprise as another optional feature, namely means for receiving temperature related information from the analyzing means and for adjusting the power output of the heating means in order to reach and maintain a predefined temperature.

When the biosensor device further comprises cooling means, the biosensor device may further comprise as another optional feature, namely means for receiving temperature related information from the analyzing means and for adjusting the operation of the cooling means in order to reach and maintain a predefined temperature.

When, the biosensor device comprises cooling/and or heating means, the biosensor substrate may further comprise as another optional feature two or more area and the heating and/or cooling means may be adapted to independently control the temperature of each of said two or more area of the biosensor substrate. As another optional feature, the heating and/or cooling means may comprise two or more heating means and/or two or more cooling means adapted to control independently the temperature of two or more area of the substrate. This is advantageous because some area may require more or less heating/cooling to achieve a same temperature. This therefore permits to obtain an homogeneous temperature in all area of the biosensor substrate, e.g. substantially across the whole surface of the biosensor substrate if a large number of areas are defined across its surface.

As another optional feature in the case when the biosensor substrate comprises two or more area, the biosensor device may further comprise means for receiving temperature related information for each of said two or more area of the biosensor substrate from the analysing means and adjusting the power output of the heating means and/or cooling means in order to reach and maintain a temperature predefined for each of said two or more area of the biosensor substrate. This permits for instance to homogenize the temperature across the surface of the biosensor substrate.

As another optional feature, the biosensor device may comprise means for creating movements in the sample fluid (e.g. for removing air bubbles from the substrate surface). Especially when heating and/or cooling means are present, air bubbles at the level of the substrate may cause the temperature of the substrate and/or of the fluid in the vicinity of the air bubble to be different from the temperature away from this air bubble. This can cause non-uniformity of the temperature distribution across the surface of the substrate. It is therefore advantageous to provide the biosensor device with means for creating movements in the sample fluid and e.g. removing air bubbles from the substrate surface. Such means can for instance pumping means or mixing means.

When the biosensor device comprises means for removing air bubbles, the device may further comprise, as an optional feature, a signaling means, coupled to the means for analyzing the temperature of the biosensor, for indicating to the user of the device the presence of the bubble-induced temperature non-uniformity. As another optional feature, the analyzing means may be coupled to the means for removing air bubbles in such a way as to automatically operate said means for removing air bubbles when a bubble-induced temperature non-uniformity is detected by the means for analyzing the temperature of the biosensor substrate. Similarly, the means for removing air bubbles may be coupled to the analyzing means in such a way as to stop the operation of the means for creating movements in the sample fluid as soon as the temperature uniformity is restored.

As another optional feature, the biosensor device may further comprise means for interrupting the operation of one or more functional parts of the biosensor device upon detection by the means for analyzing the biosensor substrate of a temperature non-uniformity across the biosensor substrate.

As another additional feature, the chamber may comprise a hydrogel. Said hydrogel being temperature responsive or comprising temperature indicating agents. In particular, the hydrogel can be present above the biosensor substrate and occupy all or part of the chamber. This is advantageous because it permits, in addition to the temperature control at the level of the biosensor substrate to monitor and control the temperature at the level of the solution. The temperature at various points of the solution can be monitored or controlled by placing temperature indicating agents at those points. The hydrogel being mainly composed of water (50 to 99%, preferably 70 to 97%), it does substantially not interfere with the analysis/detection, sample pre-treatment (preparation)/PCR/specific chemical reaction etc to be performed.

As an optional feature, the temperature indicators may be placed in a volume or area which smaller dimension ranges from 1 μm to 1 mm, preferably 5-500 μm, most preferably 10-300 μm.

As another optional feature, the volume or area occupied by the temperature indicators are placed (e.g. the spots) may be circular, spherical, rod-like or pillar like among others.

Another embodiment of the present invention relates to a method of producing a biosensor solid or hydrogel substrate, said method comprising providing a solid or hydrogel substrate material and incorporating into and/or at the surface of the solid or hydrogel substrate material, one or more temperature indicating agents whereby a physical property of the agents changes depending upon temperature. For example, each agent may operate by changing its optical properties, e.g. one or more optical property.

As an optional feature of this production method, a step is provided for incorporating, into and/or at the surface of the sensor substrate, e.g. of the solid or hydrogel substrate material, one or more probes able to each specifically bind one analyte molecule, e.g. target biological compound. According to this embodiment of the method, both the one or more probes and the one or more temperature indicating agents may advantageously be applied by ink-jet printing.

Linking of the temperature indicating agent to the porous solid or hydrogel substrate can be realized by reaction (e.g. polymerization), preferably by photoreaction (e.g. photopolymerization) allowing well defined patterned deposition of the temperature indicating agent.

Another embodiment of the present invention relates to a method of analysis of a sample fluid suspected of containing one or more analyte molecules such as target biological compounds, said method comprising:

a) analyzing a sensor substrate such as a biosensor solid or hydrogel substrate comprising one or more probes able to each specifically bind one analyte molecule such as a target biological compound and one or more temperature indicating agents each operating by changing a physical property such as an optical property with temperature, so as to gain temperature-related information from the one or more temperature indicating agents,
b) contacting the sample fluid with the sensor substrate, e.g. biosensor solid or hydrogel substrate, and
c) analyzing the sensor substrate, e.g. biosensor solid or hydrogel substrate after contacting the sample fluid so as to determine the presence and/or the concentration of the one or more analyte molecules, e.g. target biological compounds.

As an optional feature when the analyte molecules are tagged with fluorescent moieties, the analytical method of the invention may further include after step (c), another step wherein the sample fluid is removed while increasing temperature. In this embodiment, the decreasing fluorescence signal as a function of increasing temperature may provide additional information about the concentration of a specifically bounded analyte.

As an other optional feature, the analytical method of the invention may further include a pre-heating step of the sensor substrate, e.g. the biosensor solid or hydrogel substrate, in order to raise its temperature up to a desirable temperature, e.g. a temperature within the range from about 20 to about 95° C., this pre-heating step preferably occurring prior to step (a). This feature is advantageous because it permits to perform the method of analysis at the temperature providing the higher binding specificity between the probes and the target biological compounds. Pre-heating may also be useful in a preparation step, e.g. if the analyte comprises double stranded DNA that needs to be separated prior to perform the analysis or if an enzyme used in the detection process needs to be cleaved to take its active form.

As another optional feature, the analytical method of the present invention may further comprise a PCR step wherein the temperature of the substrate is cycled a predetermined number of time prior to step (a). In particular, the temperature may be cycled between a first temperature comprised between 94° C. and 98° C., a second temperature comprised between 50 and 64° C. and a third temperature comprised between 70 and 74° C.

In another embodiment, the present invention relates to a device for performing a PCR, said device comprising:

a recipient for receiving the biological molecular species to be amplified, said recipient comprising a hydrogel, said hydrogel being temperature responsive or comprising one or more temperature indicating agents,

heating and/or cooling means, and

means for analyzing said hydrogel so as to retrieve temperature-related information from said one or more temperature indicating agents.

The hydrogel may occupy all of part of the chamber. This is advantageous because it permits to monitor and control the temperature at the level of the solution. The temperature at various points of the solution can be monitored or controlled by placing temperature indicating agents at those points. The hydrogel being mainly composed of water, it does not prevent the PCR amplification to be performed. A feedback process (as described above) can be implemented in order to automatise the temperature control.

In the following the present invention will mainly be described with respect to a solid or hydrogel substrate but the present invention is not limited thereto and includes any suitable substrate within its scope.

Also the present invention will mainly be described with reference to biological target compounds but the present invention is not limited thereto but may include any suitable analyte molecules.

The temperature indicating agents will mainly be described with reference to agents which change their optical properties with temperature. However, the present invention is not limited thereto and includes agents which change any suitable physical property with temperature such as electrical conductivity, magnetic susceptibility, its volume or dimension in order to exert mechanical force or pressure or a change in shape.

Further, the present invention will now be described by reference to a micro-array assay, but the person skilled in the art understands that the invention is not limited thereto and may advantageously be applied as well to any suitable sensing technique of which ELISA tests or Western/Southern/Northern Blot are only examples.

In one embodiment, the present invention relates to a biosensor solid or hydrogel substrate for the analysis of a sample fluid suspected of containing one or more target biological compounds which may be such as, but not limited to, the following:

oligopeptides having from about 5 amino-acid units to about 50 amino-acid units,

polypeptides having more than 50 amino-acid units,

proteins including enzymes,

oligo- and polynucleotides,

antibodies, or fragments thereof,

RNA, and

DNA.

For certain target biological compounds, a denaturation step may be beneficial prior to analysis, e.g. double stranded DNA can be separated into single strands in order to allow specific binding of the single strands to the probes present in and/or on the surface of the biosensor solid or hydrogel substrate. Such a denaturation step can be implemented in a convenient manner for instance by heating the sample fluid. When the sample fluid is heated in such a denaturation step, an optional cooling step may be performed in order to keep the strands separated.

The target biological compounds are preferably tagged with labels that permit their detection. These labels can be luminescent (e.g. fluorescent, phosphorescent, chemiluminescent or electroluminescent), radioactive, enzymatic, calorimetric, sonic (e.g. resonance of micro-bubbles) or magnetic labels. Specifically bindable ligands can be used in place of a label, in which case the ligand is bound in a next step with a compatible label-bearing agent. Preferably, the labels used to tag the target biological compounds are optically detectable such as luminescent or calorimetric labels.

Suitable fluorescent or phosphorescent labels for instance include, but are not limited to, fluoresceins, Cy3, Cy5 and the like. Suitable chemiluminescent labels for instance include, but are not limited to, luminol, cyalume and the like. Suitable radioactive labels for instance include, but are not limited to, isotopes like ¹²⁵I or ³²P. Suitable enzymatic labels for instance include, but are not limited to, horseradish peroxidase, beta-galactosidase, luciferase, alkaline phosphatase and the like. Suitable calorimetric labels for instance include, but are not limited to, colloidal gold and the like. Suitable sonic labels for instance include, but are not limited to, microbubbles and the like. Suitable magnetic beads for instance include, but are not limited to, Dynabeads™ and the like.

Each target biological compound can be tagged with a significant number of labels, e.g. up to about 300 identical labels (during an eventual PCR amplification step for instance) in order to increase sensibility of the method. As an optional step, unbound labels not incorporated into the target biological compound and still present in the sample fluid may be removed from the sample fluid, if necessary, by means of one or more chemical and/or physical treatments (e.g., but not limited to, chemical PCR purification, dialysis or reverse osmosis) in order to reduce any background signal during later measurements.

The sample fluid suspected of containing one or more target biological compounds can be from industrial or natural origin. Examples of sample fluids suitable for performing the method of this invention may be, but are not limited to, body fluids such as sputum, blood, urine, saliva, feces or plasma from any animal, including mammals (especially human beings), birds and fish. Other non-limiting examples include fluids containing biological material from plants, nematodes, bacteria and the like. For a suitable performance of the method of this invention, it is preferred that said biological material is present in a substantially fluid form, more preferably a liquid form, for instance in the form of a solution in a suitable dissolution medium. The volume of the sample fluid to be used in the method of this invention is not a limiting parameter of the invention and can for instance take any value between about 5 μl and 1 ml, preferably between about 50 μl and 400 μl.

In many cases, it is desirable to incorporate a buffer (e.g. a hybridization buffer) either directly into the sample fluid to be analyzed or as an integral part of the detection unit (e.g. added as a fluid or in lyophilized form either above or below the biosensor solid or hydrogel substrate), thus eliminating the need for a separate hybridization buffer storage area.

The biosensor substrate may be a solid material capable to bind specifically with some biological species. As used herein, the term “solid” should be understood as opposed to liquid or gaseous. Hydrogels therefore may be considered as a particular solid form as well. Some substrate materials have to some degree an inherent capacity to bind one or more kind of biological compounds (e.g. nylon affinity for DNA or RNA biopolymers), but specificity for one particular biological compound usually requires certain modification of the substrate material (e.g. by attaching probes onto or into the material). The precise nature of the substrate material is not a limitative feature of the present invention and therefore can be any material already described in the art as a suitable material for biomolecule immobilization on a substrate. Non-limitative examples of such materials typically include:

organic polymers such as polyamide homopolymers or copolymers (e.g. nylon such as non-woven nylon), thermoplastic fluorinated polymers (e.g. polyvinylidene fluoride PVDF), polyvinylhalides (e.g. polyvinylchloride PVC), polysulfones, cellulosic materials (such as paper, nitrocellulose or cellulose acetate), polyolefins, polyacrylamides such as poly(N-isopropyl acrylamide), polyglycolic acid (PGA), polylactic acid (PLA), Polyglactin 910 (Vicryl®) (=copolymer of glycolic acid and lactic acid), polygluconate (Maxon™) (=Polygluconate glycolide(90)/trimethylene carbonate(10)), polydioxanone (PDS), poly-4-hydroxy butyrate and polymers from natural sources such as agarose, and hyaluronan and blends thereof in any proportions, and

inorganic materials such as glass, quartz, silica, silver, gold, aluminum, other silicon-containing materials (e.g. silicon oxide or nitride), metal oxide materials such as aluminum oxides, and the like.

materials from nature (with some treatment steps well known to the person skilled in the art) such as nitrocellulose membranes etc.

For some particular embodiments, the capture probes are attached to particle substrates in the nanometer or micrometer range. In that case the temperature sensor can also be applied on the same particles to perform the measurement in-situ, as near as possible to the capturing probes.

In some cases, it can be useful to combine more than one substrate material, e.g. by forming a layer, preferably a relatively thin layer of a first substrate material onto a second substrate material. This type of combination permits to benefit from a combination of different properties, e.g. the mechanical properties of the second layer and the chemical properties of the first layer simultaneously.

Prior to the attachment of probes, the substrate material can be inactivated, non-activated or can be activated on at least part of their surface. If activated, activation can be performed by means of a chemical treatment and/or a physical treatment, according to knowledge standard in the art. Suitable means of activation include, but are not limited to, plasma treatment, corona treatment, UV treatment or flame treatment, and chemical modification. Depending upon the kind of substrate material, suitable chemical modifications include, but are not limited to, introduction of quaternary ammonium ions (e.g. into polyamides), solvolysis (e.g. hydrolysis), derivatization of amide groups to amidine groups (e.g. in polyamides), hydroxylation, carboxylation or silylation, introduction of thiols on noble metal surfaces such as gold and silver, and/or introduction of functionalized silanes onto an oxidic surface such as glass and aluminum oxide. If inactivated, local inactivation can be suitably performed by applying blocking substances or agents, such as but not limited to salmon sperm, skim milk, or polyanions to the surface of the substrate material.

The substrate material can be non-porous or can exhibit a certain degree of porosity. If a non-porous material is used, the binding of the biological species is the result of the free diffusion of the target biological compound from the sample fluid towards the surface of the biosensor solid or hydrogel substrate. In this case, several hours of hybridization time may be required to obtain sufficient binding. This technique is usually called a “flowing over” technique. If the substrate material is porous, the binding of the target biological compound is the result of the free or forced flow of the sample fluid one or more times through the surface, i.e. either from the lower surface to the upper surface or from the upper surface to the lower surface, of the biosensor solid substrate. This embodiment can significantly shorten the time necessary for the hybridization process to occur. This technique is usually called a “flowing through” technique. In order to flow the sample fluid more than one time through the substrate material, the sample fluid can be cycled a number of times by pumping it repeatedly through the biosensor solid or hydrogel substrate.

Porous biosensor solid substrates may include a network having a plurality of pores, openings and/or channels of various geometry and dimensions. Porous biosensor solid substrates may be nanoporous or microporous, i.e. the average size of the pores, openings and/or channels may suitably be comprised between about 0.05 μm and about 10.0 μm. In one embodiment this average pore size may be between 0.1 μm and 3.0 μm. In another embodiment, the average pore size may be between about 0.2 and 1 μm. In the sense of the present invention, the term “porosity” especially means or includes the ratio of the volume of all the pores or voids in a material with respect to the volume of the whole material. In other words, porosity is the proportion of the non-solid volume to the total volume of material. In the sense of the present invention porosity is especially a fraction between 0% and 100%, e.g. ranging from 40% to 98%. In the sense of the present invention, the term “open porosity” (also called effective porosity) especially means or includes the fraction of the total volume in which fluid flow is effectively taking place.

According to an embodiment of the present invention, the inner surface area of the solid, porous substrate material is by a factor X larger than the size of this area, whereby the factor X is >100. According to another embodiment, the factor X is >1000, according to an alternative embodiment X is >10000, and according to yet another embodiment, X is >100000.

The thickness of the biosensor solid substrate is not a limiting feature of this invention and it can vary from 1 nanometer up to about 3 μm or higher, e.g. up to 1 mm. If the membrane is free-standing, e.g. in the case of a flow-through device the thickness can be from 1 micrometer to hundreds of micrometers, e.g. from 20 μm to 400 μm, or from 50 μm to 200 μm. In the case of a membrane that is provided with the probe molecules, then the membrane thickness can be much lower than specified above, e.g. from 1 nanometer to hundreds of micrometers. In the example of a device using surface plasmon resonance, which could be a flow-over device, the membrane may be a very thin silver membrane in order to allow read-out from the backside of the membrane.

The shape and or size of the substrate, e.g. the membrane, are not considered to be limiting features of the present invention. It may be circular, e.g. with a diameter ranging between about 3 and 15 mm, but any other substrate shape (rectangular, square, oval, . . . ) and/or size can apply.

The probes used for the present invention should be suitably chosen for their affinity to the target biological compounds or to the relevant modifications of said target biological compounds suspected to be present in the sample to be analyzed. For example, if the target biological compounds are DNA, the probes can be, but are not limited to, synthetic oligonucleotides, analogues thereof, or specific antibodies. A non-limiting example of a suitable modification of a target biological compound is a biotin substituted target biological compound, in which case the probe may bear an avidin functionality.

In a particular embodiment of the present invention, several different probes are deposited into and/or onto the substrate. In a more specific embodiment, multiple different probes are spotted in an array fashion on physically distinct locations along one surface of said solid substrate in order to allow measurement of different target biological compounds in parallel. This embodiment is usually named a micro-array.

In order to more easily support subsequent detection and identification, one or more additional spots (e.g. for intensity calibration and/or position detection) can be spotted as well onto the surface of the substrate material. Spotting can be suitably effected by any methods known in the art such as, but not limited to, ink-jet printing, piezoelectric spotting, robotic contact printing, micropipetting, and the like.

Following spotting, the probes become immobilized onto the surface of the substrate material, either spontaneously due to the substrate (e.g. membrane) inherent or acquired (e.g. via activation) properties, or through an additional physical treatment step (such as, but not limited to, cross-linking, e.g. through drying, heating or through exposure to a light source).

Once the probes have been deposited (e.g. via ink-jet spotting) onto a surface of the substrate material, the addition of an effective amount of a blocking agent in order to inactivate the non-spotted areas of the substrate may be helpful to prevent unspecific binding of target biological compounds or unbound labels to unspotted areas (that would likely lead to unwanted background signals) and to therefore increase the signal/noise ratio. Examples of suitable blocking substances or agents include, but are not limited to, salmon sperm, skim milk, or polyanions in general.

In another embodiment of the present invention, different labels can be used simultaneously to simultaneously measure:

(i) one or more target biological compounds from different sample fluids (e.g. different sample fluids like blood and sputum or different sample fluids originating from different locations), or
(ii) differential expression of analytes from multiple sample fluids (e.g. a sample originating from a treated patient vs. a sample originating from an untreated healthy patient, etc. . . . ), or
(iii) different types of target biological compounds from the same sample fluid (e.g. analysis of a blood sample fluid for its DNA and RNA content).

Before contacting the sample fluid with the biosensor solid substrate, heating the sample fluid to a defined temperature may be desirable to allow, through imparting more stringent binding conditions, a more precise control of the binding properties, especially binding specificity. This heating step can be achieved by heating either the biosensor substrate (e.g. a membrane) or the sample fluid or both. After the desired temperature has been reached, the sample fluid is then contacted with the substrate.

An important feature of the present invention is depositing, into and/or onto the biosensor solid or hydrogel substrate, one or more temperature indicating agents in order to permit an improved control of the temperature, and the homogeneity thereof, on the surface of the solid substrate. Providing several areas marked by temperature indicating agents on the surface of the biosensor substrate permits to assess the homogeneity of the temperature on this surface. Retrieving this information permits either to take appropriate measures for correcting this homogeneity of temperature or to take it into account when effecting the analysis. These one or more temperature indicating agents can be either embedded into the biosensor solid or hydrogel substrate (e.g. incorporated during the biosensor solid substrate material production or, if the biosensor solid substrate is a porous substrate, deposited into the pores of the biosensor solid substrate) or deposited on the biosensor substrate as a layer or as spots. If the one or more temperature indicating agents are deposited on the biosensor substrate as a layer, leading to a continuous distribution, this deposition can be made by any method known in the art such as, but not limited to, solvent casting from solution, spin coating, spraying, blade coating, painting, dip coating, screen printing and the like. If the biosensor solid substrate is non-porous, the layer of temperature indicating agents is preferably deposited before the application of the probes. If the biosensor solid substrate is porous, the layer of temperature indicating agents may be deposited either before or after the application of the probes. In the case of a porous biosensor solid substrate, the person skilled in the art understands that the layer of temperature indicating agents may diffuse to some extent inside the biosensor solid substrate. If the one or more temperature indicating agents are deposited on the biosensor substrate as spots leading to a discontinuous distribution, the spotting method used can be any spotting method known in the art, and preferably the same method as the method used to spot the probes. Most preferably this method is ink-jet printing. Independently of the porosity of the biosensor solid substrate, the spots may be deposited either before or after the application of the probes. In the case of a porous biosensor solid substrate, the person skilled in the art understands that the spots may diffuse to some extent inside the biosensor solid substrate.

The temperature indicating agent used in this invention operates by changing its optical properties at a temperature within a range between about 20° C. and 95° C. In the case of protein microarrays, a useful range is between 35 and 40° C. In the case of DNA microarrays, a useful range is 42-65° C. and another useful range (especially during the washing step) is 60-95° C. The selection of the most appropriate temperature indicating agent may depend upon parameters such as the sample fluid to be analyzed, in particular the target biological compounds contained therein. The change in optical property should preferably be detectable within a short temperature interval, e.g. not more than 5° C., preferably not more than about 3° C., more preferably not more than about 1° C. and most preferably not more than 0.5° C. In the case of liquid crystalline temperature indicating agents, this change will usually occur within a sharper interval of temperatures when the purity of liquid crystal materials is higher. Such an increase of purity usually goes together with an increase of cost. A balance must therefore be found, for each specific type of analysis, between cost and precision. Typically, the change of optical property is observed when a given range of temperatures is reached and/or passed, the measurement of this property permits therefore to determine whether the temperature of the biosensor solid substrate is above or under this temperature range. Non-limitative examples of temperature indicating agents usable in the present invention are thermochromic dyes, photochromic dyes (e.g. dispersed in a polymer matrix), liquid crystals (LCs) and temperature responsive polymers. Thermochromic dyes are chemical compounds showing a change of color (usually between a colorless and a colored form) upon a certain change of chemical or physical environment (typically a change of pH). One or more thermochromic dyes is (are) usually enclosed within microcapsules together with a dissociable salt, a weak acid and/or an appropriate solvent. Other type of mixtures using bases instead of acids are also known in the art. When the solvent is solid, i.e. below its melting temperature, the dye exists in its uncolored form, while when the solvent melts, the salt dissociates, the pH inside the microcapsule lowers, the dye becomes protonated, its chemical structure changes, and its absorption spectrum therefore shifts. Suitable thermochromic dyes comprise, but are not limited to, spirolactones, fluorans, spiropyrans, and fulgides. An example of spirolactone is crystal violet lactone depicted below.

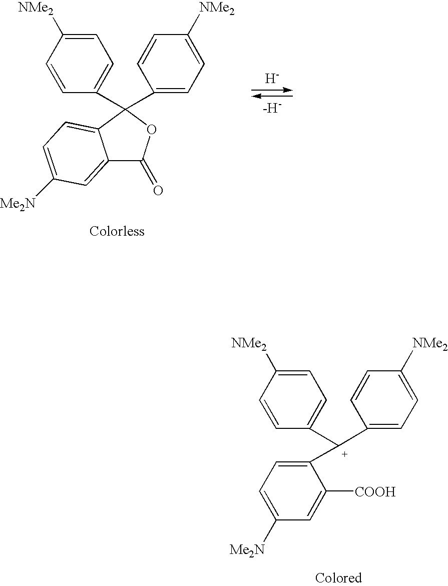

Suitable weak acids include bisphenol A, parabens, 1,2,3-triazole derivatives and 4-hydroxycoumarin, and act as proton donors, thus changing the dye molecule from its uncolored form to its protonated colored form; stronger acids would make the change irreversible. These thermochromic dyes can be used in combination with other pigments or dyes producing a color change between the color of the base pigment or dye and the color of the protonated form of the thermochromic dye. Thermochromic dyes are available over a whole temperature range between about −5° C. to 60° C., and in a wide range of colors. The color change usually happens within a 3° C. interval.

A second class of dyes are the photochromic dyes. For these dyes the rate constant for the photochromic processes are strongly dependent on the amount of free volume in the polymer matrix, and therefore, they are strongly dependent on the temperature. For sterically hindered photochromic compounds the free volume in a polymer matrix below T_{g} will be insufficient for isomerization reactions of the dye. Above the glass transition temperature of the polymer there will be a significant increase in the rate constants of the photochromic processes as a result of the increased free volume and optical changes occur.

A preferred class of thermochromic materials are liquid crystals. In most cases, liquid crystals change from a liquid crystalline light scattering state to an isotropic transparent state above a distinctive transition temperature. This can be used to indicate a temperature as for instance shown in U.S. Pat. No. 5,686,153. Low molecular weight liquid crystals can be deposited into or onto the biosensor solid substrate surface and, when heated up above a certain temperature, can provide an observable transition between a more or less transparent state (depending on the matching of the refractive indexes between the biosensor solid substrate and the isotropic phase of the liquid crystal used) and a scattering state. An advantage of using low molecular weight liquid crystals is that the temperature range over which the phase transition occurs is relatively small. The adaptation of this temperature range is done for instance by blending different liquid crystals. A drawback of low molecular weight liquid crystals is the diffusion of these liquid crystals through or across the biosensor solid substrate and a certain degree of water uptake by these liquid crystals. Other temperature indicating agents usable in the present invention are polymeric liquid crystals (PLCs). They have the advantage to be more easily printed on the biosensor solid substrate, to have a lesser tendency to diffuse through or across the biosensor solid substrate and to have a lesser tendency to absorb water. Drawback of most polymeric liquid crystals is that the temperature range over which the phase transition occurs is larger than for small molecular weight liquid crystals. The temperature related information retrieved from these polymers may therefore not be precise enough for certain types of analysis. Among the class of polymeric liquid crystals, side-chain liquid crystals with a siloxane polymer backbone are preferred. A particular example is given in FIG. 1.

The polymeric liquid crystal of FIG. 1 is a siloxane-polystyrene block-copolymer, containing cyanobiphenyls side groups at the siloxane chains providing a liquid crystalline phase. This particular polymer is in a smectic phase (i.e. a phase in which long range orientation order exist and where the liquid crystalline moieties are grouped into layers) below 80° C. and has a relatively sharp transition within 5° C. around 80° C. showing a change from scattering to clear. This polymer is stable and resistant to water uptake. The transition temperature can be adjusted for instance by selecting other mesogens (i.e. the fundamental unit of a liquid crystalline material that induces structural order) than the cyanobiphenyls moieties or by varying the end groups or spacer groups. The most useful range of temperature is comprised between 20 and 95° C. Liquid crystalline siloxane rings (FIG. 2) have usually a sharper temperature range for the liquid-to-crystal liquid transition than the linear polysiloxane because of their narrower molecular weight distribution. In the example of FIG. 2, this transition occurs within a temperature interval of about 1° C. The general formula of a preferred series of liquid crystalline siloxane rings is depicted below:

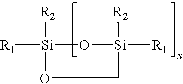

wherein x is 3 or 4, and wherein R₁ is alkyl (such as —CH₃, —C₂H₅), cycloalkyl (such as cyclohexyl) or phenyl. Non limitative examples of mesogen R₂ groups include, but are not limited to, the following:

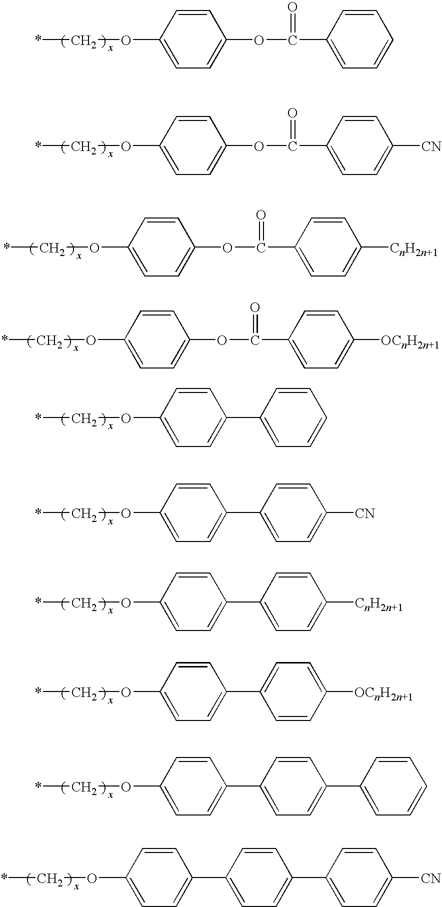

wherein x is preferably between 2 and 12 and wherein n is preferably between 1 and 6.

In another embodiment of the present invention, the polymeric, oligomeric or cyclic liquid crystal may be blended with a monomer or dissolved therein. The solution or blend is subsequently deposited onto the biosensor solid substrate and polymerized. This permits to avoid any contact between the liquid crystalline material and water and/or the sample fluid. The system forms then a so-called polymer-dispersed liquid crystal which is distributed as droplets or channels in a polymer matrix that forms a barrier against the sample fluid. Suitable monomers for this purpose include, but are not limited to, acrylates, methacrylates, epoxides and mixtures thereof, which can be cured either thermally or by means of light irradiation.

An alternative way to prevent contact of the sample fluid with the liquid crystalline material is encapsulation of liquid crystal droplets within a polymer shell. Liquid crystal filled polymer capsules can for example be obtained by emulsifying a mixture of a polymer, a liquid-crystalline material and an organic solvent totally or partially miscible with water (such as, but not limited to, dichloroethane) in water. A stabilizer such as polyvinyl(alcohol) can also be added. The organic solvent dissolves in the water phase and subsequently evaporates. As a result, the polymer and the liquid crystal in the emulsion droplets are no longer miscible and a polymer shell is formed at the interface with the water phase. In order to improve the homogeneity of the size distribution of the LC-filled polymer capsules in the mixture of polymer, LC and solvent can be injected drop-wise in the water by making use of, for instance, an ink-jet nozzle. In the case of the use of ink-jet nozzles, capsule diameters may be in the 2-20 micron range. After removal of water, the capsules can be dispersed in a monomer system and the resulting blend can be locally deposited by making use of a printing technique (such as, but not limited to, ink-jet printing). The monomer-system can thereafter be cured by photo-polymerization. The advantage of this method is the possibility to use low molecular weight liquid crystals without a risk of water uptake or diffusion problems.

FIG. 5 shows two photographs taken by optical microscopy. Both photographs represents two LC-filled polymer capsules at room temperature. The left panel shows them between two crossed polarizers and the right panel shows them between two parallel polarizers. These capsules have a diameter of about 7 μm. At a certain temperature above room temperature (here at 35° C.), the contrast disappears in both panel and the capsules becomes invisible/transparent (not shown in FIG. 5). In another embodiment of the present invention, an enhancement of the visibility of the transition from the scattering state to the isotropic state is achieved by the addition of a small amount of a dye to the liquid crystal system. The dye concentration should be chosen to make the dye hardly visible in the isotropic phase but clearly visible in the scattering phase. This is possible because the light pathway is longer in the latter. An advantage of this embodiment is that a single detection means can be easily used to detect both the liquid crystalline-to-liquid transition and the location of the target biological compounds (if those target biological compounds are tagged with optically visible markers such as, but not limited to, fluorescent markers). In a special design the dye-liquid crystal composite can be printed above an already printed region containing another dye with a contrasting color. For instance, a liquid crystal composite containing a blue dye can be printed on a substrate containing a red dye.

In another embodiment the dye is a fluorescent dye. In this embodiment, the fluorescence is much more intense when the liquid crystalline material is in its liquid crystalline phase (e.g. nematic or smectic (i.e. no positional order, but long-range orientation order)). Here also, a single detection means can be easily used to detect both, the liquid crystalline-to-liquid transition and the location of the target biological compounds. So in this preferential case the fluorescent dye is exited with the light used also to excite the fluorescent target biological compounds and detected by the detection means (e.g. a microscope, photodetector, photodetector array, camera such as CCD or CMOS camera, etc.).

In another embodiment of the present invention, the liquid crystalline material itself is intrinsically colored or fluorescent.

In another embodiment different liquid crystalline materials having different liquid crystalline-to-liquid transition temperatures are deposited at different locations of the same substrate. In this embodiment, a more precise idea of the exact temperature can be assessed rather than merely concluding on the fact that the biosensor solid substrate is either above or under a certain critical temperature.

In another embodiment of the present invention, two LC materials with a slightly different temperature transition are printed next to each other. One LC material has a transition slightly below the desired temperature and the other one has a transition temperature slightly above this temperature. By analyzing the two elements with the detection means (e.g., a microscope, a photodetector, a CMOS or CCD camera, etc.), the temperature can be accurately controlled between the two extremes given by the transition temperatures of these two different LC materials. Also the temperature indicating agents can be distributed over the surface of the biosensor solid substrate in order to obtain information about the temperature distribution over this surface.

In another embodiment of the present invention, cholesteric liquid crystals are used as temperature indicating agents. Cholesteric liquid crystals are rod-like liquid crystals having a liquid-crystalline phase in which the molecules are closely aligned within a distinct series of layers, with the axes of the molecules lying parallel to the plane of the layers and with the orientation of molecules in adjacent layers being slightly rotated. Because the LC molecules of a particular layer are always slightly rotated in one direction (e.g. clockwise) when compared to the LC molecules presents in the layer just below them, each inter-layers column of LC molecules describes a helix in the direction perpendicular to the molecules. Because of the molecular anisotropy, the uniaxial optical indicatrix also describes a helix into the same direction and reflection of light will occur when Bragg's conditions are met. The reflection wavelength λ relates to the helicoidal pitch p as follow:

λ= n̅·p,

wherein n̅ is the average refractive index. The pitch p is temperature dependent and consequently, so is the reflection wavelength. The pitch becomes especially temperature sensitive when the cholesteric LC molecules are selected to exhibit a smectic phase at temperatures lower than the temperature wished to be monitored. The smectic phase unwinds the helix when the transition is approached and the color changes very steeply with temperature. Simple thermochromic coatings can be made by dispersing the cholesteric material in a polymer binder such as a polyurethane or by microencapsulating the liquid crystal and disperse the capsules in a polymeric binder. These dispersions can be applied from solution as a film on the substrate and dried or cured. Best results are obtained when the films are applied on a black biosensor solid substrate.

The temperature information can be read from the reflection color. In this case spectral analysis will give the best results. A suitable analyzing means would for instance be a CCD camera provided with a color filter array.

In another embodiment of the present invention, temperature responsive materials undergoing a phase transition (this includes melting, crystalline-amorphous, LCST or other transitions) may be used as temperature indicating agents.

For instance, (hydrophilic) polymers, co-polymers or hydrogels exhibiting a lower critical solution temperature (LCST) may be used as temperature indicating agents. These polymers, co-polymers or hydrogels switch from a transparent to a scattering state above the LCST. Non limitative examples of temperature responsive polymers includes polymers, co-polymers or hydrogels based on one or more of the following monomers: N-substituted acrylamides (such as N-alkylacrylamides, as N-isopropylacrylamide, di(m)ethylacrylamide, carboxyisopropylacrylamide, hydroxymethylpropylmethacrylamide, etc), acryloylalkylpiperazine and N-vinylcaprolactam as well as co-polymers thereof with hydrophilic monomers such as but not limited to hydroxyethyl(meth)acrylate, (meth)acrylic acid, acrylamide, polyethyleneglycol(meth)acrylate, N-vinyl pyrolidone, dimethylaminopropylmethacrylamide, dimethylaminoethylacrylate, N-hydroxymethylacrylamide or mixtures thereof, and/or co-polymerized with hydrophobic monomers such as but not limited to (iso)butyl(meth)acrylate, methylmethacrylate, isobornyl(meth)acrylate, glycidyl methacrylate or mixtures thereof. Example of useful polymers are poly(N-isopropyl acrylamide) (LCST=32° C.), poly(N,N′-diethyl-acrylamide) (LCST=25 to 35° C.) and poly(-N-acryloyl-N′-alkylpiperazine) (LCST=37° C.). The N-substituted acrylamides may be copolymerized with for instance oxyethylene, trimethylol-propane distearate, ε-caprolactone and mixture thereof among others.

Temperature responsive hydrogels may be made for instance by mixing one or more N-substituted acrylamide monomers with an effective amount of one or more crosslinkers. Suitable examples of crosslinkers include, but are not limited to, N-methyl-bisacrylamide and poly(ethyleneglycol) diacrylate. The molar ratio monomer:crosslinker may suitably be in the range between 1:25 and 1:1000. Furthermore, an initiator (either a photo-initiator or a thermal initiator) may be added in order to initiate polymerization (e.g. in a 1 to 5 wt % ratio with respect to the monomer).

The one or more monomers may be mixed with an aqueous solvent (typically between 50 and 90% by weight H₂O or a H₂O/methanol mixture) and the mixture is then deposited onto the biosensor substrate (e.g. a membrane) for instance by means of ink-jet printing and is subsequently polymerised. Preferably polymerization takes place immediately after deposition such that the mixture may be fixed onto the biosensor substrate (e.g. a membrane) and does not diffuse significantly into the biosensor substrate (e.g. a membrane) before polymerization. In order to enhance contrast prior to the deposition of the hydrogel, a dye or a polymer containing a dye may be deposited.

According to an embodiment of the present invention, at least one of the temperature indicating agent is a temperature responsive (hydrophilic) polymers, co-polymers or hydrogels having a crosslink density of 0.002 to 1, preferably 0.05 to 1.

Also, an agent delivering an optical signal can be deposited prior to the deposition of the hydrogel or can be embedded in the hydrogel. This agent can for instance be a fluorescent die or fluorescent beads such as fluorescent microspheres (e.g. polystyrene microspheres bearing dyes). One way to detect the temperature change would in this case be to monitor the fluorescence intensity change resulting from the clear-scattering transition of the hydrogel when the lower critical solution temperature (LCST) is reached. Another way to detect the temperature change would be to measure the transmission, scattering or reflection of light.

The temperature control means can be any means such as, but not limited to, a thermostatic fluid bath, thermostatic air circuit, resistance heater, Peltier device, and the like. In some embodiments, the temperature control means can be two or more temperature control means (i.e. two or more heating means and/or cooling means) adapted to independently control the temperature of two or more area across the biosensor substrate and more in particular across the biosensor substrate surface. As an example, a two-dimensional array of a plurality of heater and/or cooler elements can be provided below or on the biosensor substrate. Each of these elements can for instance be coupled to one or more control terminal enabling an active matrix to change the state (on or off) of each element individually. The use of multiplexing or passive matrix techniques are other, although less preferred, alternatives.

In a particular embodiment of this invention, a feedback process may be implemented in which a controller receives the signals corresponding to the temperature readings, and adjusts power output to the temperature control means in order to maintain the selected temperature. If two or more heating means and/or cooling means are provided to independently control the temperature of two or more area across the biosensor substrate, a feedback process may be implemented in which a controller receives the signals corresponding to the temperature readings at each area, and adjusts power output to the temperature control means of each area in order to maintain selected temperature in each area, e.g. the same temperature in all area. Analysis of the substrate in the final step of the method of the invention may be performed via an optical set-up comprising an epi-fluorescence microscope and a CCD camera or any other kind of optical detection device of which a camera is only one possibility. Other possibilities include photodetectors or a microscope. This optical set-up preferably comprises a (preferably UV) light source capable of exciting the labels at their respective excitation wavelength, in the case of fluorescent or phosphorescent labels. Other detection methods are usable as well.

The detection of chemiluminescent labels may for instance be performed by adding an appropriate reactant to the label and observing its fluorescence via the use of a microscope.

The detection of radioactive labels is for instance performed by placing a medical X-ray film directly against the solid substrate, which film develops as it is exposed to the label and creates dark regions which correspond to the emplacement of the probes of interest.

The detection of enzymatic labels is for instance performed by adding an appropriate substrate to the label and observing the result of the reaction (e.g. color change) catalyzed by the enzyme.

The detection of calorimetric labels is for instance performed by adding an appropriate reactant to the label and observing the resulting appearance or change of color.

The detection of sonic microbubble labels is for instance performed by exposing said labels to sound waves of particular frequencies and recording the resulting resonance.

The detection of magnetic beads is for instance performed by means of one or more magnetic sensor(s).

The preferred detection method is the use of an optical detector such as a CCD camera, CMOS camera, microscope or photodetector, etc.

FIG. 3 presents a scheme of a particular set-up usable in the method of the present invention. In a housing (10), a sample fluid (4) is represented in a chamber (1) and pressure is applied at the inlet (3). This pressure forces the sample fluid (4) downwards through the porous biosensor solid substrate (2). A glass plate (7) permits the analysis of the biosensor solid substrate (2) to be, if desired, optically performed. A means (5) is present for analyzing the biosensor solid substrate (2) so as to determine the presence of one or more target biological compounds. A means (6) is present for analyzing the biosensor solid substrate (2) so as to gain information concerning the temperature of the biosensor solid substrate. The dashed line is there to indicate that both means (5) and (6) are eventually a single means (e.g. an optical detection means such as a CCD camera or any other kind of optical detection device of which a camera is only one possibility. Other possibilities include photodetectors or a microscope.). Provision may be made to cycle the sample fluid (4) back to chamber 1 after it has passed through substrate (2). Preferably, the substrate is continuously or intermittently but regularly wetted with the sample fluid. In this way representative temperatures of the sample fluid may be obtained.

FIG. 4 presents a scheme of a biosensor solid substrate (2) according to one embodiment of the present invention on which probes (8) and temperature indicating agents (9) have been printed at particular locations of the biosensor solid substrate.

### EXAMPLES

### Example 1

The polymer of FIG. 1 was dissolved in xylene and printed at specific locations of a nylon biosensor membrane by using an ink-jet printer. The solvent was thereafter allowed to evaporate. The transition temperature from scattering to clear was then observed around 80° C. with a charge-coupled device (CCD) camera.

### Example 2

In another example we mixed 65 parts by weight of a polymer (commercially available from Merck under the name LCP93; degree of polymerization n+m≈40; smectic to isotropic transition according to Merck at 97° C.)

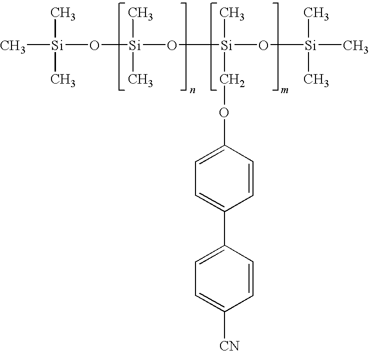

with 35 parts by weight of ethoxylated bisphenol-A diacrylate (Sartomer 349, commercially available from Sartomer). Both materials have a similar refractive index of around 1.55 avoiding light scattering when the liquid crystal polymer LCP093 is heated above its liquid crystalline transition temperature. For curing the sample is blended with 2 parts by weight photoinitiator (Irgacure 651—Ciba Specialty Chemicals). The mixture forms a paste that can be printed by means of a PDMS mould on the biosensor substrate. Curing proceeds by illumination with a UV source PL10 lamp (Philips—365 nm light at an intensity of 0.6 mW·cm-²). The sample changes its appearance from highly scattering to clear transparent when heated above 74° C. The transition proceeded between 73 and 75° C.

### Example 3

The liquid crystal molecule of FIG. 2 was mixed with the bis-acrylate of ethoxylated bisphenol-A and subsequently printed at specific locations of a nylon biosensor membrane by using an ink-jet printer. UV light was used to photopolymerize the mixture in the presence of a photoinitiator (2 wt % Irgacure 651 commercially available from Ciba Specialty Chemicals). The transition temperature from scattering to clear was then observed between 60 and 61° C. with a charge-coupled device (CCD) camera.

### Example 4

A mixture of a biocompatible polymer (poly-(lactic-co-glycolic)acid, PLGA), a liquid crystal (n-pentylcyanobiphenyl) and dichloroethane (DCE) as a solvent was injected in water (PLGA:LC:DCE ratios being 0.05:0.20:99.75, and 0.3% by weight polyvinyl alcohol being added to H₂O as a stabilizer to prevent the emulsion droplets from coalescing) through ink-jet nozzles and LC-filled polymer capsules where obtained. After removal of water, the capsules were dispersed in a monomer system (ethoxylated bisphenol-A+2 wt % Irgacure 651 from Ciba Specialty Chemicals) and the resulting blend was locally deposited on a nylon biosensor substrate by making use of ink-jet printing and cured by photo-polymerization of the monomer system. The transition temperature from scattering to clear was then observed at 35° C. and occurred within a temperature interval of not more than 1° C.

### Example 5

In this example, a temperature indicating agent having a transition temperature centered on 37° C. is provided.

First, a blend is made containing the following components:

50.3 wt-% of a liquid crystal mixture,

48.0 wt-% of a blend of reactive monomers, and

1.75 wt-% of a photoinitiator.

The liquid crystal mixture used contains two materials:

25 parts by weight:

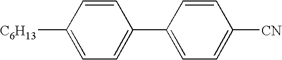

75 parts by weight:

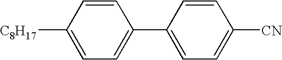

The blend of reactive monomers contains the following two materials:

75 parts by weight:

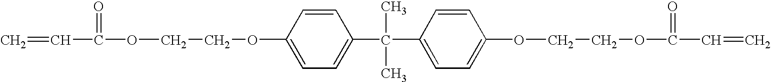

and 25 parts by weight:

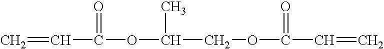

The photoinitiator was Irgacure 651 from Ciba Specialty Chemicals.

The mixture was applied by inkjet printing and photopolymerized by UV light. After polymerization the liquid crystal phase separates from the polymer matrix and is light-scattering. When heated above 37° C., the printed dots become highly transparent because the liquid crystal mixture goes through its phase transition to isotropic.

### Example 6

The same procedure as in example 5 was followed except that the blend further incorporated a dye and therefore contained the following components:

50.0 wt-% of the same liquid crystal mixture

48.0 wt-% of the same blend of reactive monomers

1.75 wt-% of the same photoinitiator, and

0.25 wt-% of a dye

The dye was a sulphoindocyanine fluorescent dye (structure shown below) known as Cyanine-5.18 which emits at 667 nm:

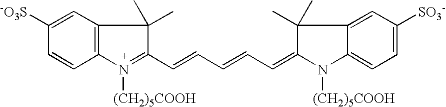

Cyanine-5.18

When excited with light of 650 nm or below the printed dots fluoresce. When heated above 37° C., the fluorescent intensity suddenly drops by an order of magnitude because the printed dots become highly transparent and non-scattering.

### Example 7

In this example, a temperature indicating agent was made with a transition temperature of about 61° C. The transition operates within a temperature interval of not more than 1° C.

In this case the same basic composition has been chosen as given in example 4. However the liquid crystal has been replaced with E7, a commercial mixture containing the following materials:

51 parts by weight n-pentylcyanobiphenyl,

25 parts by weight n-heptylcyanobiphenyl,

16 parts by weight n-octyloxycyanobiphenyl, and

8 parts by weight n-pentylcyanoterphenyl.

The mixture was deposited by inkjet printing and then photopolymerized by means of UV light. After polymerization the liquid crystal phase separates from the polymer matrix and is light-scattering. When heated above 61° C., the printed dots become highly transparent because the liquid crystal mixture goes through its phase transition to isotropic.

### Example 8

The same procedure as in example 7 was followed except that the blend further incorporates a dye and therefore contains the following components:

50.0 wt-% of the same liquid crystal mixture as in example 6,

48.0 wt-% of the same blend of reactive monomers as in example 6,

1.75 wt-% of the same photoinitiator as in example 6, and

0.25 wt-% of a dye.

The dye is a red-fluorescent dye (λₑₓ 630 nm; λₑₘ 670 nm) (described in J. R. Fries et al. (2001) Chimica Oggi, 19, 18) having the following formula:

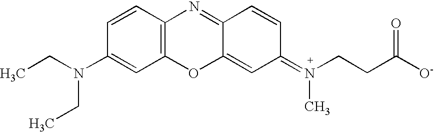

When excited with light of 630 nm, the printed dots fluoresce. When heated above 61° C., the fluorescent intensity suddenly drops visibly because the printed dots become highly transparent and non-scattering.

### Example 9

A solution of 49 wt % (N-isopropylacrylamide (NIPA) (monomer)/diethylene glycol diacrylate), and 1 wt % Irgacure 2959+50 wt % methanol was made. The mole ratio N-isopropylacrylamide:diethylene glycol diacrylate was 180:1. To this solution fluorescent beads (Crimson™ microspheres having a diameter of 0.02 μm) were added. The solution was printed at specific locations of a nylon biosensor membrane and polymerized under UV radiation. After the rinsing of the hydrogel in water (=removing methanol), the transition temperature (LCST) was observed around 32° C. via a change in fluorescence intensity recorded with a charge-coupled device (CCD) camera.

### Example 10

Hydrogels were prepared by following the same procedure as in example 9 except that the nature of the monomer, additional comonomer and cross-linker was according to table 1 below:

| | | | LCST |
|---|---|---|---|
| Monomer | Comonomer | Crosslinker | (° C.) |
| VCL | DMAA | MBA | 22.9 |
| (N-Vinylcaprolactam) | | | |
| NIPA | / | A-HPC | 32.2 |
| | | (hydroxypropyl | |
| | | cellulose) | |
| VCL | GMA | MBA | 32.8 |
| | (Glycidyl | | |
| | methacrylate) | | |
| NIPA | | ACR-CELL | 34.1 |
| NIPA | VCL | MBA | 36.7 |
| NIPA | VP (N-vinyl | ACR-CELL | 39.9 |
| | pyrolidone) | | |
| DMAA | GMA | MBA | 47.1 |
| (dimethylacrylamide) | | | |
| NIPA | VP | MBA | 48.6 |
| NIPA | DMAPMA | MBA | 54.6 |
| | (dimethylaminopropyl | | |
| | methacrylamide) | | |
| NIPA | HMAA | ACR-CELL | 55.2 |
| | (N-(hydroxymethyl)- | | |
| | acrylamide) | | |
| VCL | DMAPMA | MBA | 58.0 |

The LCST temperatures were measured by recording the change in turbidity.

### Example 11

Solution were prepared comprising 250 mg N-isopropylacrylamide (NIPA), 5 mg diethylene glycol diacrylate (DEGDA), 15 mg Irgacure 2959 (photoinitiator), poly ethylene glycol diacrylate (PEGA) (comonomer), and 80% water was made. The mole ratio of PEGA was varied form 0 to 9% with respect to NIPA. FIG. 6 shows the sudden change in optical signal during temperature increase referred to as LCST, observed after polymerization of said solutions. By increasing the ratio of PEGA, the LCST could be varied between 36 and 47° C.

1. A non-porous or porous biosensor substrate made of solid or hydrogel material comprising one or more temperature indicating agents, each of said one or more temperature indicating agents operating by changing its optical properties and being deposited in and/or at the surface of the biosensor solid or hydrogel substrate as one or more layers and/or spots.

2. A non-porous or porous biosensor substrate according to claim 1, further comprising one or more probes able to each specifically bind one target biological compound.

3. A non-porous or porous biosensor substrate according to claim 2, wherein said one or more probes form an array of probe locations present in and/or at the surface of the biosensor solid or hydrogel substrate.

4. A non-porous or porous biosensor substrate according to claim 1, wherein the change in optical properties of said one or more temperature indicating agents occurs at a temperature comprised between 20° C. and 95° C.

5. A non-porous or porous biosensor substrate according to claim 1, wherein the change in optical properties of said one or more temperature indicating agents is detectable within a temperature interval of not more than 3° C.

6. A non-porous or porous biosensor substrate according to claim 1, wherein said one or more temperature indicating agents are deposited in and/or at the surface of the biosensor solid substrate as one or more spots, said biosensor solid substrate further comprising one or more probes able to each specifically bind one target biological compound, wherein said one or more probes form an array of probe locations present in and/or at the surface of the biosensor solid substrate, and wherein said spots are located at places distinct from the location of said one or more probes.

7. A non-porous or porous biosensor substrate according to claim 1 wherein at least one of said one or more temperature indicating agents comprises a liquid crystalline material.

8. A non-porous or porous biosensor substrate according to claim 1, wherein at least one of said one or more temperature indicating agents comprises one or more side-chain liquid crystal with a siloxane polymer backbone and/or one or more liquid crystalline siloxane rings.

9. A non-porous or porous biosensor substrate according to claim 1, wherein at least one of said one or more temperature indicating agents comprises a temperature responsive polymer, co-polymer or hydrogel that is able to undergo a change in optical property upon heating.

10. A non-porous or porous biosensor substrate according to claim 9, wherein said change results from a phase transition.

11. A non-porous or porous biosensor substrate according to claim 9, wherein said change is a change from a transparent state to a scattering state.

12. A non-porous or porous biosensor substrate according to any of claim 9, wherein said temperature responsive polymer, co-polymer or hydrogel is based on one or more N-substituted acrylamides.

13. A non-porous or porous biosensor substrate according to claim 1, wherein said substrate comprises a first substrate material and a second substrate material forming a layer onto the first material.

14. A non-porous or porous biosensor substrate according to claim 1, wherein at least one of said one or more temperature indicating agents contains or is deposited onto a coloring agent.

15. A biosensor device comprising
a chamber (1) including a porous or non-porous biosensor substrate (2) made of a solid or hydrogel material, said biosensor substrate (2) comprising one or more temperature indicating agents (9) operating by changing their optical properties,
inlet means (3) for introducing a sample fluid (4) suspected to contain one or more target biological compounds into said chamber (1) in such a way that said sample fluid (4) contacts said biosensor substrate (2),
means (5) for analyzing said biosensor substrate (2) after said sample fluid (4) having contacted said biosensor substrate (2) so as to determine the presence and/or the concentration of said one or more target biological compounds onto said biosensor substrate, and
means (6) for analyzing said biosensor substrate (2) so as to retrieve temperature-related information from said one or more temperature indicating agents (9).

16. A biosensor device according to claim 15, further comprising one or more probes (8) able to each specifically bind one of said one or more target biological compounds.

17. A biosensor device according to claim 15, wherein said sample fluid (4) contacts said biosensor substrate (2) by flowing through said biosensor substrate (2).

18. A biosensor device according to claim 15, wherein said means (5) for the determination of the presence of said one or more target biological compounds and said means (6) for retrieving temperature-related information from said one or more temperature indicating agents are the same means.

19. A biosensor device according to claim 15, further comprising heating means for raising the temperature of the sample fluid (4) and/or the biosensor substrate (2) and/or cooling means for decreasing the temperature of the sample fluid (4) and/or the biosensor substrate (2).

20. A biosensor device according to claim 19, wherein said biosensor substrate comprises two or more areas and wherein said heating means and/or cooling means are two or more heating means and/or two or more cooling means adapted to independently control the temperature of each of said two or more areas of the biosensor substrate (2).

21. A biosensor device according to claim 15 wherein said chamber (1) further comprises a hydrogel, said hydrogel being temperature responsive or comprising one or more temperature indicating agents.

22. A biosensor device according to claim 15 further comprising:
A recipient for receiving the biological molecular species to be amplified, said recipient comprising a hydrogel, said hydrogel being temperature responsive or comprising one or more temperature indicating agents, and or
A pre-treatment chamber comprising or not a hydrogel, said hydrogel being temperature responsive or comprising one or more temperature indicating agents.

23. A method of producing a porous or non-porous biosensor substrate made of a solid or hydrogel material according to claim 1, comprising providing a solid or hydrogel substrate material and incorporating into and/or at the surface of said solid or hydrogel substrate material one or more temperature indicating agents operating by changing their optical properties.

24. A method of analysis of a sample fluid suspected of containing one or more target biological compounds comprising the steps of:
a) analyzing a porous or non-porous biosensor substrate made of a solid or hydrogel material and comprising one or more temperature indicating agents, said one or more temperature indicating agents operating by changing their optical properties, to gain temperature-related information from said one or more temperature indicating agent,
b) contacting said sample fluid with said biosensor substrate, and
c) analyzing said biosensor substrate after contacting said sample fluid so as to determine the presence and/or the concentration of said one or more target biological compounds.

25. A method according to claim 24, wherein said biosensor substrate is pre-heated prior to step (a).

26. A method according to claim 25, wherein the temperature of the biosensor substrate is raised up to a temperature comprised between 20° C. and 95° C.

27. A method according to claim 24, wherein the temperature of the substrate is a changed according to a pre-defined regime for performing a PCR reaction.

28. A device for performing a PCR, said device comprising:
A recipient for receiving the biological molecular species to be amplified, said recipient comprising a hydrogel, said hydrogel being temperature responsive or comprising one or more temperature indicating agents,
Heating and/or cooling means, and
means for analyzing said hydrogel so as to retrieve temperature-related information from said one or more temperature indicating agents.
